(19) United States
(12) Patent Application Publication
(10) Pub. No.: US 2010/0189674 A1
(43) Pub. Date: Jul. 29, 2010

(54) Petrolatum Having Silicone-Like Properties

(75) Inventors: Brett Davis Morrison, Grove City, PA (US); Anthony Paul Banaszewski, Petrolia, PA (US); Timothy D. Miles, Petrolia, PA (US); Timothy A. Yasika, Petrolia, PA (US); Geraldine Ibanez Humphreys, Petrolia, PA (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: Sonneborn Inc., Mahwah, NJ (US)

(21) Appl. No.: 12/599,020

(22) PCT Filed: May 8, 2008
(86) PCT No.: PCT/US2008/063036

§ 371 Date: Mar. 11, 2010

Related U.S. Application Data
(60) Provisional application No. 60/917,378, filed on May 11, 2007.

(51) Int. Cl. A61K 8/31; A61Q 5/12

(52) U.S. Cl. 424/70.1

(57) ABSTRACT

Novel petrolatums having silicone-like properties are disclosed. Silicone-free compositions containing the novel petrolatums also are disclosed.

### CROSS-REFERENCE TO RELATED APPLICATIONS

This application claims the benefit of U.S. provisional patent application No. 60/917,378, filed May 11, 2007.

### FIELD OF THE INVENTION

The present invention is directed to novel petrolatums having the sensory properties of a silicone fluid. The present invention also is directed to silicone-free compositions that possess the benefits of a silicone-containing composition, while avoiding disadvantages associated with silicone fluids.

### BACKGROUND OF THE INVENTION

Petrolatums, also known as petroleum jellies, are homogenous mixtures of oily and waxy, long-chain, nonpolar hydrocarbons. Petrolatum is a mixture of hydrocarbons of a nature such that when the melted material is cooled to ordinary room temperatures, the mixture congeals to a translucent, amorphous or jelly-like material. Petrolatums are odorless and tasteless, range in color from white to yellow, and typically differ from one another in consistency and shear strength.

The U.S. Pharmacopeia (U.S.P.) uses the terms white petrolatum and white petroleum jelly interchangeably and describes them as a purified mixture of semi-solid hydrocarbons obtained from petroleum and wholly or nearly decolorized. Petrolatum is further defined as having a melting point range of 38° C. to 60° C. (100.4° F. to 140° F.) and as having a consistency of not less than 100 and not more than 300 dmm, as determined by the U.S.P. official method. A lesser decolorized grade is described in the National Formulary (N.F.) as a “yellow” grade and as free or nearly free from odor and taste.

Petrolatums have been used beneficially in a variety of personal care, pharmaceutical, and industrial applications for more than 100 years. Typically, petrolatums used in cosmetic, food, personal care, and pharmaceutical applications meet FDA and U.S.P. requirements. Petrolatums add lubricity and moisture resistance to lotions, creams, ointments, and hand cleaners, for example. As a result, cosmetic, personal care, and pharmaceutical formulators often choose petrolatums as a formulation base.

Food processors rely on petrolatums for uses ranging from baking and candy-making to packaging. In jar candles, the addition of petrolatum affects the crystallinity of the waxes to give a smoother, more pleasing appearance. In metal polishes and buffing compounds, petrolatums protect against moisture and corrosion.

Among the many attributes of petrolatum, which make its use in topical applications advantageous, are its moisturizing efficacy, water barrier property, water repellency, resistance to being washed off by water, physiological inertness, and chemical inertness and stability. The hydrating properties of petrolatums set a standard against which other moisturizers are compared.

The chief drawbacks of petrolatum in topical compositions are greasiness, cosmetic inelegance, and an inability to provide a thin, consistent film over a large area of skin. Silicone fluids do not possess these drawbacks, and, in fact, the benefits of silicone fluids are a good afterfeel, cosmetic elegance, and spreadability to provide a thin, consistent film.

Therefore, although petrolatums are available in a variety of grades having a wide range of properties and are widely-used, a need exists in the art for an improved petrolatum. Present day petrolatums have the drawbacks discussed above, including lacking the beneficial physical and sensory properties of silicone fluids. Present day petrolatums therefore cannot be substituted for silicone fluids in personal care, cosmetic, and pharmaceutical compositions. Such a substitution would be useful in the art because silicone fluids are expensive and coming under closer regulatory scrutiny. The present invention is directed to petrolatums useful as economical and efficacious substitutes for silicone fluids.

### SUMMARY OF THE INVENTION

The present invention is directed to a novel class of petrolatums that can be used as a substitute for silicone fluids. Accordingly, an aspect of the present invention is to provide petrolatums that melt at skin temperature, have a low heat of fusion, a consistent feel, a hydrocarbon range of C₁₀ to C₉₀, an average molecular weight of about 300 to about 450, a drop melting point of about 100° F. to about 125° F., a consistency (cone penetration) of about 250 to 300 dmm (dmm is decimillimeter or 0.1 millimeter), and adynamic viscosity at 25° C. of about 100,000 to about 200,000 centipoise (cps). The present petrolatums therefore meet USP requirements.

Another aspect of the present invention is to provide petrolatums having at least one of the following physical properties:

(a) a flow onset of about 45° C. or less;

(b) a storage modulus at 0.1 Pa (G′=0.1 Pa) of about 50° C. or less; and

(c) a complex viscosity at 0.1 Pa·s (|η*|=0.1 Pa·s) of about 45° C. or less.

In preferred embodiments, the petrolatums exhibit at least two of the physical properties (a), (b), and (c). In more preferred embodiments, the petrolatums exhibit each of the physical properties (a), (b), and (c).

Yet another aspect of the present invention is to provide petrolatums that exhibit the sensory properties of a silicone fluid, yet are free of silicone compounds.

Another aspect of the present invention is to provide compositions containing a petrolatum of the present invention. In preferred embodiments, the composition is free of a silicone fluid, but provides the benefits of a silicone fluid-containing composition.

These and other novel aspects of the present invention will become apparent from the following nonlimiting detailed description of the preferred embodiments.

### BRIEF DESCRIPTION OF THE FIGURES

FIG. 1 contains plots of viscosity (Pa·s) vs. temperature (° C.) showing the flow onset temperature of present day commercial petrolatums at 25 Pa·s;

FIG. 2 contains plots of viscosity (Pa·s) vs. temperature (° C.) showing the flow onset temperature of inventive petrolatums at 25 Pa·s;

FIG. 3 contains plots of viscosity (Pa·s) vs. temperature CC) showing the flow onset temperature difference between the inventive petrolatum of Example 1 and Penreco® Ointment Base™ No. 4;

FIG. 4 contains plots of G′(Pa) vs. temperature (° C.) showing the storage modulus of present day commercial petrolatums at Pa=0.1′;

FIG. 5 contains plots of G′(Pa) vs. temperature (° C.) showing the storage modulus of inventive petrolatums at Pa=0.1′;

FIG. 6 contains plots of G′(Pa) vs. temperature (° C.) showing the storage modulus difference between the inventive petrolatum of Example 1 and Penreco® Ointment Base™ No. 4;

FIG. 7 contains plots of |η*|(Pa·s) vs. temperature (° C.) showing the complex viscosity of present day commercial petrolatums at 0.1 Pa·s;

FIG. 8 contains plots of |η*|(Pa·s) vs. temperature (° C.) showing the complex viscosity of inventive petrolatums at 0.1 Pa·s; and

FIG. 9 contains plots of |η*|(Pa·s) vs. temperature (° C.) showing the complex viscosity difference between the inventive petrolatum of Example 1 and Penreco® Ointment Base™ No. 4.

### DETAILED DESCRIPTION OF PREFERRED EMBODIMENTS

The present petrolatums are novel compositions that possess properties making them suitable as substitutes for silicone fluids in a variety of practical applications. In particular, the present petrolatums possess the following properties, which typically are associated with a silicone fluid:

skin benefits: imparts a soft, silky feel, excellent spreading, no oily residue or build-up, detackification, non-greasy feel, compatibility with cosmetic ingredients, and transient emolliency; and

hair benefits: good wet combing, transient shine, and no build-up.

These properties make a present petrolatum excellent for replacing silicone fluids, in whole or in part, in cosmetic, personal care, and pharmaceutical compositions.

A present petrolatum has “silicone-like” attributes that combine the known advantages of a petrolatum with the easy handling and sensory feel of a white mineral oil. Accordingly, a present petrolatum can be used as a complete or partial replacement for silicone fluids and mineral oils in cosmetic, personal care, and pharmaceutical formulations.

present petrolatum is a soft, translucent-white, high gloss product having a soft, cushiony feel, and a velvety, powdery afterfeel. The advantageous properties of a present petrolatum include: suitability for mineral and silicone oil free formulations, melting at skin temperature, excellent spreadability, easy handling, no oily residue, non-greasy feel, low tack, and a short, average, or long play time. “Playtime” is the length of time a compound or composition, after application to skin, remains on the skin prior to evaporation and/or absorption.

The present petrolatums exhibit a low melt point and soft consistency, which contribute to a soft silky feel, while providing cushion, emolliency, lubricity, and non-sensitizing properties. The combination of softness and relatively low melting point results in a present petrolatum that melts at skin temperature, while exhibiting an excellent spreadability and a low tack. Such positive properties are not exhibited by present day petrolatums.

More particularly, a class of petrolatums that can be used as a substitute for silicone fluids in cosmetic, personal care, and pharmaceutical applications has been discovered. The petrolatums comply with the purity requirements for petrolatum, as specified in both 21 CFR 172.880 and the USP monograph for petrolatums, and have silicone-like properties.

A petrolatum of the present invention comprises:

about 5 to about 20% saturated straight chain hydrocarbons with a carbon number distribution of about C₂₀-C₄₅;

0 to about 20% saturated microcrystalline hydrocarbons with a carbon number distribution of about C₃₀-C₉₀; and

about 60 to about 85% saturated branched and cycloparaffinic hydrocarbons with a carbon number distribution of about C₁₀-C₅₀.

The present petrolatums have the following physical properties:

drop melting point (ASTM D127): about 100° F. to about 125° F.,

cone penetration (ASTM D937): about 240 to about 300 dmm, and

viscosity @ 25° C. (Dynamic Viscometer): about 100,000 to about 200,000 cps.

In preferred embodiments, the petrolatum has a drop melting point of about 105° F. to about 120° F. and/or a cone penetration of about 270 to about 300 dmm.

Other preferred petrolatums of the present invention also exhibit one or more of the following properties:

viscosity (ASTM D445): about 3.5 to about 10.0 centistokes (cst) at 210° F.,

color, Saybolt (ASTM D156): >+22,

average molecular weight: about 300 to about 450, and

molecular weight range: about 200 to about 1400.

Petrolatums of the present invention also have demonstrated the following properties:

| | |
|---|---|
| Congealing Point, ° F. | about 90 to about 98 |
| Viscosity @ 210° F., SUS | about 38 to about 42 |
| Loviband Color, 3″ cell | about 0.1 to about 0.6 |
| Specific Gravity @ 60° C./25° C. | about 0.80 to about 0.83 |
| Saybolt Color | about +23 to about +32 |
| Carbon Number @ 5% point | about 14 to about 22 |
| Viscosity @ 130° F., cSt | about 10 to about 13 |
| Viscosity @ 130° F., SUS | about 60 to about 68 |
| Refractive Index @ 130° F. | about 1.4510 to about 1.4550 |
| Cloud on heating, ° F. | about 90 to about 140 |
| Cloud on cooling, ° F. | about 85 to about 135 |

A present petrolatum having silicone-like properties is useful in a variety of cosmetic, personal care, and pharmaceutical applications where an emollient and/or moisturizer is required or desired, for example, hand/body lotions, scalp protectors, massage creams, hair conditioners, body washes, and suncare and sunscreen products.

Three inventive petrolatums have the properties summarized in Table I. In Table I, the present petrolatums are compared to three present day, commercial USP petrolatums:

TABLE I
Physical Properties

| | | | | | Commercial USP Petrolatums | | |
|---|---|---|---|---|---|---|---|
| | | | | | Unilever | | Penreco ® |
| | | | | | Vaseline ® | Penreco ® | Snow White ™ |
| | | | | | Petroleum | Ointment | Petrolatum, |
| | Method | Example 1 | Example 2 | Example 3 | Jelly | Base ™ 4 | USP |
| Congealing Point | ASTM D938 | 95.0 | 93.0 | 92.0 | 124.2 | 103.0 | 120.0 |
| Drop Melting Point | ASTM D127 | 108.0 | 105.3 | 115.0 | 137.8 | 124.5 | 130.8 |
| Cone Penetration | ASTM D937 | 298 | 273 | 278 | 178 | 270 | 200 |
| Lovibond Color, | IP 17 | 0.6 | 0.3 | 0.2 | 6.4 | 0.5 | 0.9 |
| 3″ cell | | | | | | | |
| Viscosity @ 130° F., cSt | ASTM D445 | 11.26 | 11.88 | 10.98 | N/A | N/A | N/A |
| Viscosity @ 210° F., cSt | ASTM D445 | 3.95 | 4.38 | 4.01 | 7.2 | 7.22 | 12.9 |
| Specific Gravity @ | ASTM D1298 | 0.821 | 0.824 | 0.815 | 0.829 | 0.832 | 0.837 |
| 60° C./25° C. | | | | | | | |
| Saybolt Color | ASTM D156 | 24 | 26 | 30 | <10 | 22 | 17 |
| Carbon Number @ 5% | IP 507-4 | 19 | 19 | 15 | 22 | 20 | 28 |
| Point | | | | | | | |
| Average Molecular Weight | ASTM D2502 | 364 | 412 | 383 | 364 | 448 | 532 |
| Refractive Index @ 130° F. | ASTM D1218 | 1.4538 | 1.4557 | 1.4520 | N/A | N/A | N/A |
| Viscosity @ 25° C. | Dynamic | about | about | about | N/A | N/A | N/A |
| | Viscometer | 150,000 cps | 150,000 cps | 150,000 cps | | | |

N/A = not available

Table I shows that Penreco® Ointment Base™ 4 has physical properties similar to the inventive petrolatums. However, as demonstrated below, the differences in other physical properties between a present petrolatum and Penreco® Ointment Base™ 4 are substantial.

In particular, the petrolatums of Examples 1, 2, and 3 were compared to commercial USP petrolatums and to commercial silicone fluids. First, all compositions tested were evaluated in a “neat”, undiluted and unformulated, form using the methods set forth in ASTM E 1490. The evaluations were performed by a panel of 8 to 10 trained judges.

The results are summarized in Tables II and III. Table II compares nine sensory attributes of a present petrolatum to silicone fluids and to commercial USP petrolatums. It can be seen that the sensory attributes of the present petrolatums match well with various viscosity grades of silicone fluids used in cosmetic, personal care, and pharmaceutical formulations. Table II also shows that present day petrolatums possess sensory attributes substantially different from both the silicone fluids and the inventive petrolatums.

Silicone fluids are included in personal care and cosmetic compositions because the sensory attributes of silicone fluids are desirable to consumers. An especially important sensory attribute is “afterfeel”. Table II shows that the present petrolatums have an afterfeel comparable to silicone fluids. In contrast, present day commercial petrolatums have a “draggy, tacky” afterfeel, which is esthetically undesirable or unacceptable to consumers. This poor afterfeel is a major reason why a present day petrolatum cannot be substituted for silicone fluid in personal care, cosmetic, and pharmaceutical formulations. This problem has been overcome by the present petrolatums, and the present petrolatums can be substituted, wholly or in part, for silicone fluids, and for mineral oils, in such formulations.

TABLE II
COMPARISON OF SENSORY ATTRIBUTES

| | | | | Silicones | | |
|---|---|---|---|---|---|---|
| | | | | Dow Corning ® | | |
| | | | | 345 Fluid, | Dow Corning ® | Dow Corning ® |
| | | | | Cyclopentasiloxane | 200 Fluid, | 200 Fluid, |
| | Inventive petrolatums | | | (and) | 50 cs | 100 cs |
| | Example 3 | Example 1 | Example 2 | Cyclohexasiloxane | Dimethicone | Dimethicone |
| Absorption | High | High | High | High | High | High |
| Slip | High | High | High | High | High | High |
| Drag | Low | Low | Low | Low | Low | Low |
| Cushion | Low | Low | Medium | Low | Low | Low |
| Tackiness | Low | Low | Low | Low | Low | Low |
| Residue | Low | Low | Low | Low | Low | Low |
| Play Time | Low | High | Medium | Low | Low | Low |
| Gloss | Low | Low | High | Low | Low | Low |
| Afterfeel | Powdery soft | Soft, dry | Velvety, | Powdery | Soft | Light, |
| | | | Powdery, | | | powdery |
| | | | Smooth | | | |

| | | | | Commercial USP Petrolatums | | |
|---|---|---|---|---|---|---|
| | Silicones | | | | Penreco | |
| | Dow Corning ® | Dow Corning ® | Dow Corning ® | Unilever | ®Snow | |
| | 200 Fluid, | 200 Fluid, | 200 Fluid, | Vaseline ® | White ™ | Penreco ® |
| | 200 cs | 350 cs | 500 cs | Petroleum | Petrolatum, | Ointment |
| | Dimethicone | Dimethicone | Dimethicone | Jelly | USP | Base ™ 4 |
| Absorption | Medium | Low | Low | Low | Low | Low |
| Slip | High | Low | Low | Medium | Medium | Medium |
| Drag | Low | Low | Low | Medium | Medium | Medium |
| Cushion | Low | Low | High | Medium | Medium | Medium |
| Tackiness | Low | Low | Low | High | High | High |
| Residue | Low | Medium | Medium | High | High | High |
| Play Time | Medium | High | High | High | High | High |
| Gloss | Low | Medium | Medium | High | High | High |
| Afterfeel | Soft, Smooth | Soft, Velvety | Soft, Velvety | Draggy, | Draggy, | Draggy, |
| | | | | Tacky | Tacky | Tacky |

Table III provides a further comparison of the present petrolatums to silicone fluids and to commercial petrolatums for various properties that contribute to “afterfeel”. In this test, the “neat” compositions were evaluated by a panel of trained judges using the protocol of ASTM E1490. Table III shows that the three commercial petrolatums left a draggy and tacky afterfeel, and failed to exhibit any of the positive afterfeel attributes of light, fast absorption, smooth, soft, and cushiony. In contrast, the inventive petrolatums and silicone fluids each exhibited various positive afterfeel attributes, and no negative afterfeel attributes of drag and tack.

As expected, no silicone fluid or inventive petrolatum exhibited all of the positive afterfeel attributes. This is expected because various silicone fluids are designed to exhibit different afterfeel properties for different applications. Silicone fluids often are used in combination to provide an expanded number of positive afterfeel attributes. The petrolatums of present Examples 1-3 exhibit positive afterfeel attributes equal to, or essentially equal to, the positive attributes of corresponding silicone fluids. Accordingly, the present petrolatums can be substituted for a corresponding silicone fluid, in whole or in part. The present petrolatums also have been found useful as a substitute for mineral oils. A present petrolatum also can be admixed with additional petrolatums of the present invention and/or with silicone fluids to provide a desired array of positive afterfeel attributes.

From Table III, it can be seen that the petrolatum of Example 3 corresponds to and is an excellent substitute for cyclopentasiloxane, cyclohexsiloxane, and dimethicone fluids of 50 cs and 100 cs. The petrolatum of Example 1 corresponds to and is an excellent substitute for dimethicone fluids of 200 cs and 350 cs. The petrolatum of Example 2 corresponds to and is an excellent substitute for dimethicone fluids of 350 cs and 500 cs. As stated above, admixing petrolatums of the present invention can provide the desired properties that correspond to any present day silicone fluid or mixture of silicone fluids.

TABLE III
COMPARISON OF AFTERFEEL ATTRIBUTES (NEAT FORM)

| | | | | Silicones (Dow Corning ®) | | |
|---|---|---|---|---|---|---|
| | | | | Dow Corning ® 345 | | |
| | | | | Fluid, | Dow Corning ® | Dow Corning ® |
| | | | | Cyclopentasiloxane | 200 Fluid, | 200 Fluid, |
| | Inventive Petrolatum | | | (and) | 50 cs | 100 cs |
| | Example 3 | Example 1 | Example 2 | Cyclohexasiloxane | Dimethicone | Dimethicone |
| Light | ✓ | | | ✓ | ✓ | ✓ |
| Fast Absorption | ✓ | | | ✓ | ✓ | |
| Smooth | | ✓ | ✓ | | | |
| Soft | ✓ | ✓ | | | ✓ | |
| Cushiony | | | ✓ | | | |
| Draggy | | | | | | |
| Tacky | | | | | | |

| | | | | Commercial USP Petrolatums | | |
|---|---|---|---|---|---|---|
| | Silicones (Dow Corning ®) | | | | Penreco ® | |
| | Dow Corning ® | Dow Corning ® | Dow Corning ® | Unilever | Snow | |
| | 200 Fluid, | 200 Fluid, | 200 Fluid, | Vaseline ® | White ™ | Penreco ® |
| | 200 cs | 350 cs | 500 cs | Petroleum | Petrolatum, | Ointment |
| | Dimethicone | Dimethicone | Dimethicone | Jelly | USP | Base ™ 4 |
| Light | | | | | | |
| Fast Absorption | | | | | | |
| Smooth | ✓ | | | | | |
| Soft | ✓ | ✓ | ✓ | | | |
| Cushiony | | ✓ | ✓ | | | |
| Draggy | | | | ✓ | ✓ | ✓ |
| Tacky | | | | ✓ | ✓ | ✓ |

In addition to exhibiting sensory properties different from commercial petrolatums, the present petrolatums also exhibit physical, and particularly rheological, properties that differentiate the inventive petrolatums from commercial petrolatums. It is theorized, but not relied upon, that these differences in physical properties at least partially contribute to the improved and unexpected sensory benefits demonstrated by the present petrolatums.

One difference in physical properties is flow onset. The rheology of petrolatums is such that as the temperature increases, the viscosity of the petrolatum decreases. The term “flow onset” is defined as the temperature at which the viscosity of the petrolatum is 25 Pa·s (Pascal-second). In this test, a constant stress (Pa, pascal) was applied to a petrolatum sample together with a programmed temperature ramp to determine the flow onset temperature of the petrolatums. The instrument used in test was a TA AR2000ex Rheometer. With respect to sensory properties, flow onset correlates to how quickly the petrolatum melts on the skin.

Table IV contains flow onset temperatures, in ° C., for petrolatums of the present invention and for present day commercial petrolatums. Table IV shows that commercial petrolatums have a substantially higher flow onset temperature, i.e., about 45° C. to about 55° C., than the inventive petrolatums. The data in Table IV shows that a present day commercial petrolatum either does not melt on the skin or melts wholly or partially in time frames that are unsuitable to perform as a substitute for a silicone fluid.

TABLE IV

| | Flow Onset |
|---|---|
| | (° C.) |
| Example 3 | 31.7 |
| Example 1 | 39.7 |
| Example 2 | 36.7 |
| Ointment Base ™ No. 4 (Penreco ®) | 47.2 |
| Snow White ™ Petrolatum USP (Penreco ®) | 54.7 |
| Vaseline ® (Unilever) | 53.0 |

Petrolatums of the present invention therefore have a flow onset temperature of about 45° C. or less, and typically about 30° C. to less than about 45° C., or less than about 42° C. A preferred flow onset temperature for a present petrolatum is about 30° C. to about 40° C.

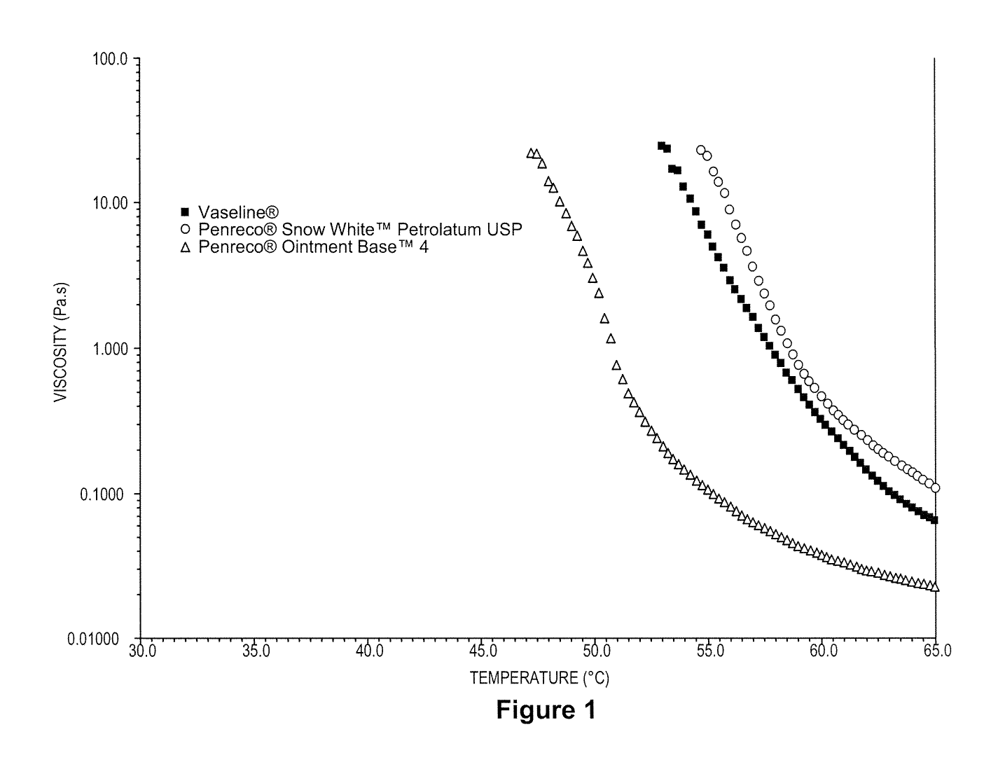

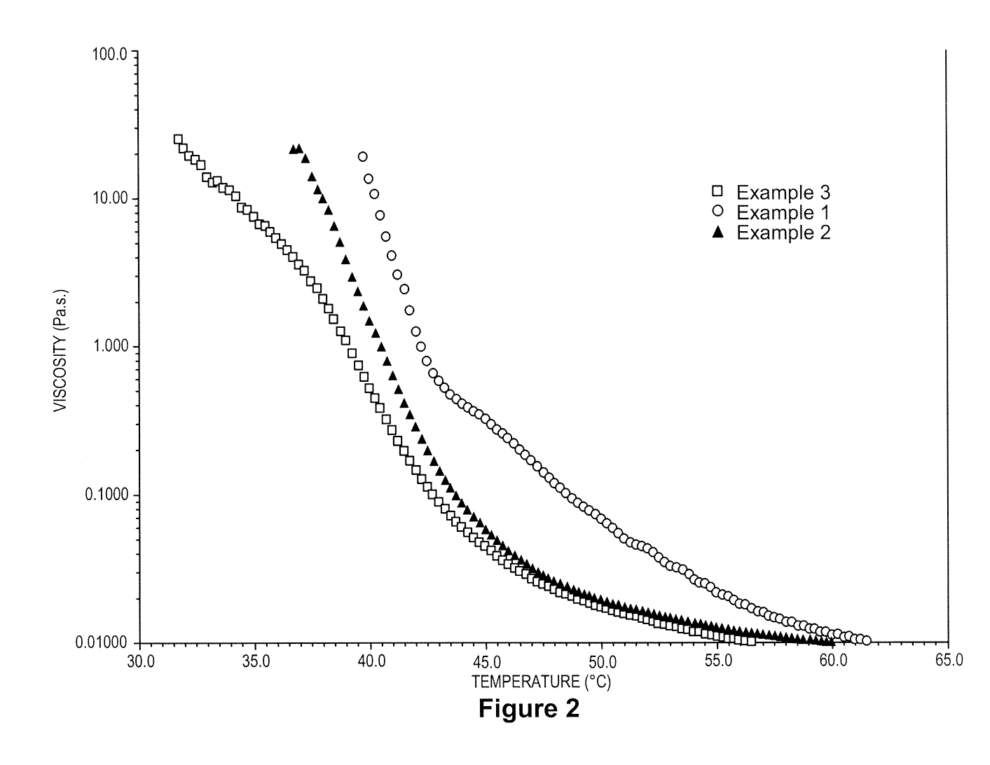

To further demonstrate the difference in flow onset temperature between a present petrolatum and present day commercial petrolatums, FIGS. 1-3 contain plots of viscosity (Pa·s) vs. temperature (° C.). FIG. 1 contains plots of present day commercial petrolatums. FIG. 2 contains plots for petrolatums of the present invention.

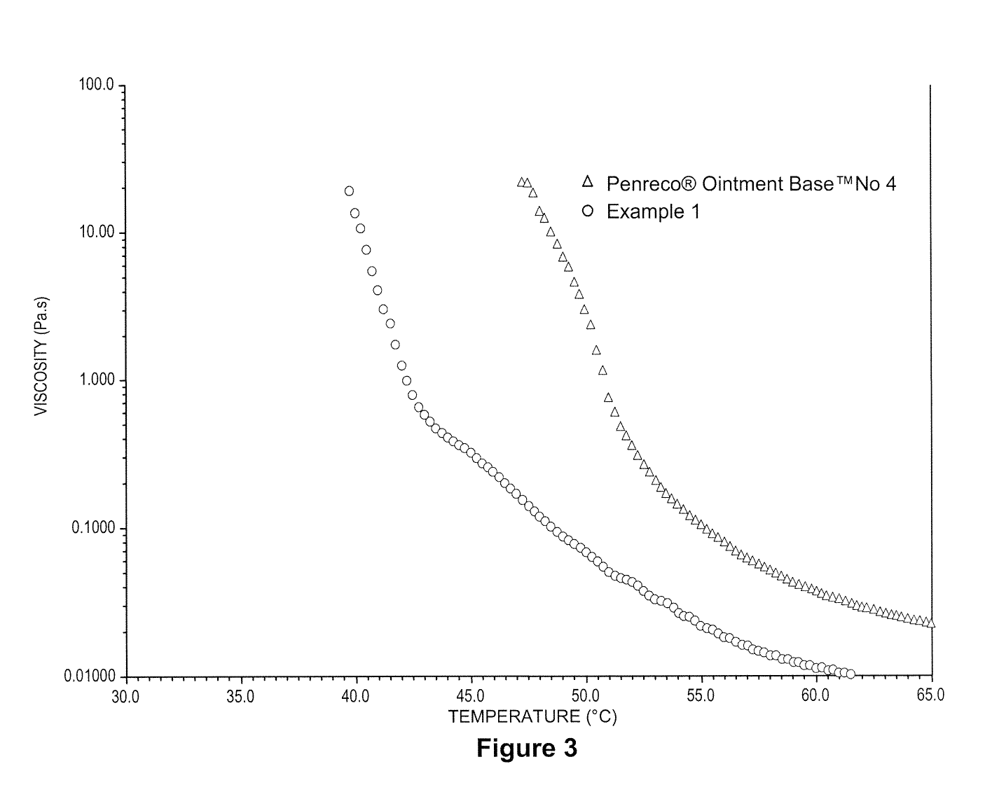

It is readily observed that the flow onset temperature for petrolatums of the present invention are substantially lower than present day commercial petrolatums. FIG. 3 contains plots of viscosity (Pa·s) vs. temperature (° C.) showing the difference in flow onset temperature between the inventive petrolatum of Example 1 and a commercial petrolatum (Penreco® Ointment Base™ No. 4) having a similar melting point and cone penetration, i.e., 108.0° F. (inventive) vs. 124.5° F. and 298 dmm (inventive) vs. 270 dmm, respectively.

Additional differences in physical properties were found based on performing an oscillatory analysis. In this test, a constant oscillatory torque at a constant frequency was applied to a test sample vs. a temperature ramp. The two properties measured were the storage modulus G*(Pa), and the complex (combined) viscosity, (|η*|(Pa·s)). In terms of sensory properties, the storage modulus correlates to the “cushion” of the petrolatum, or the energy required to initiate movement of the petrolatum, and the complex viscosity correlates to the “drag” of the petrolatum, or a measure of the petrolatum to resist flow.

Storage Modulus (G′) is equal the quotient of the shear stress divided by the shear strain multiplied by the cosine of the phase angle. i.e.,

G′=(σ₀/γ₀)cos δ

wherein
σ₀=Shear Stress (Pa)
γ₀=Shear Strain
δ=Phase Angle between Shear Stress and Shear Strain.

Complex Viscosity (|η*|) is equal to G* divided by the frequency, i.e., |η*|=G*/ω,

wherein ω=frequency (rad/s)

G′=(G′²+G″²)^{0.5}(Function of Storage Modulus and Loss Modulus)

G′=(σ₀/γ₀)cos δ
wherein
   σ₀=Shear Stress (Pa)
   γ₀=Shear Strain
   δ=Phase Angle between Shear Stress and Shear Strain

G″=(σ₀/γ₀)sin δ
wherein
   σ₀=Shear Stress (Pa)
   γ₀=Shear Strain
   δ=Phase Angle between Shear Stress and Shear Strain
      Loss Modulus (G″) is equal the difference of the shear stress divided by the shear strain times the sine of the phase angle.

The two properties of storage modulus and complex viscosity further differentiate the inventive petrolatums from prior petrolatums. The storage modulus analysis shows that the storage modulus at 0.1 Pa. i.e., G′=0.1 Pa, for present day commercial petrolatums is about 55° C. to greater than 65° C. Table V summarizes the storage modulus for petrolatums of the present invention and the present day commercial petrolatums. The data show that the present petrolatums require a lower energy to initiate movement, i.e., are spread more easily. The inventive petrolatums, therefore, overcome a disadvantage associated with prior petrolatums, i.e., spreadability over large areas of skin.

TABLE V

| | G′ = 0.1 Pa |
|---|---|
| Example 3 | 39.0° C. |
| Example 1 | 47.4° C. |
| Example 2 | 39.4° C. |
| Ointment Base ™ No. 4 | 54.6° C. |
| Snow White ™ Petrolatum USP | >65° C. |
| Vaseline ® | >65° C. |

Petrolatums of the present invention therefore have a storage modulus (G′=0.1 Pa) of about 50° C. or less, and typically about 35° C. to about 50° C., or about 48° C. Preferred petrolatums of the present invention have a G′=0.1 Pa of about 37° C. to about 48° C.

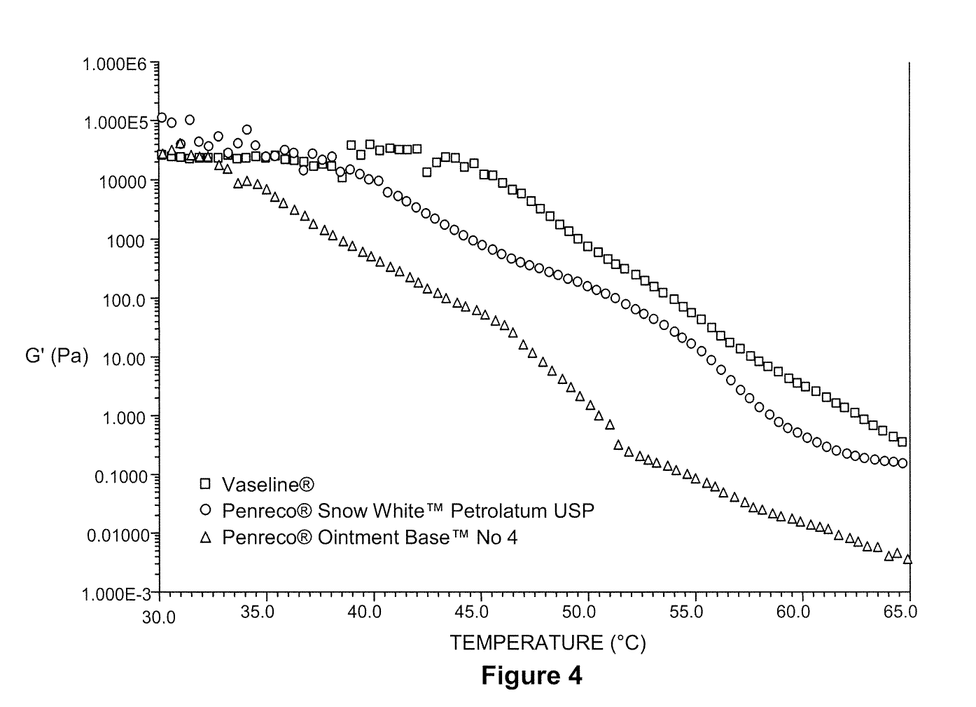

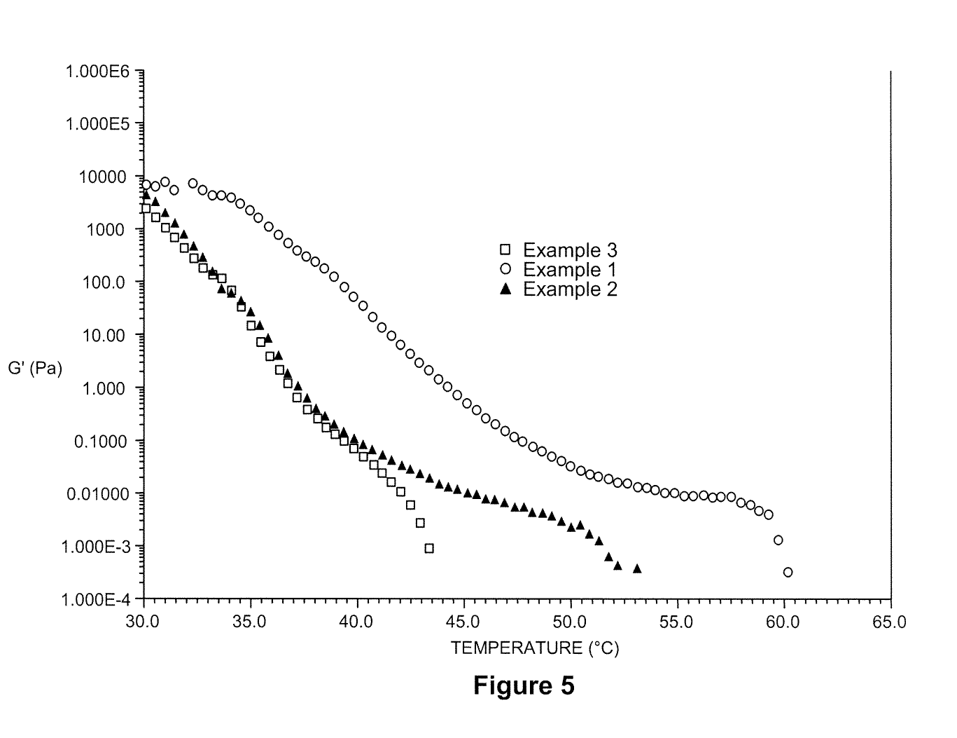

To further demonstrate the differences in storage modulus between a present petrolatum and present day commercial petrolatums, FIGS. 4-6 contain plots of G′(Pa) vs. temperature (° C.). FIG. 4 contains plots for present day commercial petrolatums. FIG. 5 contains plots of petrolatums of the present invention.

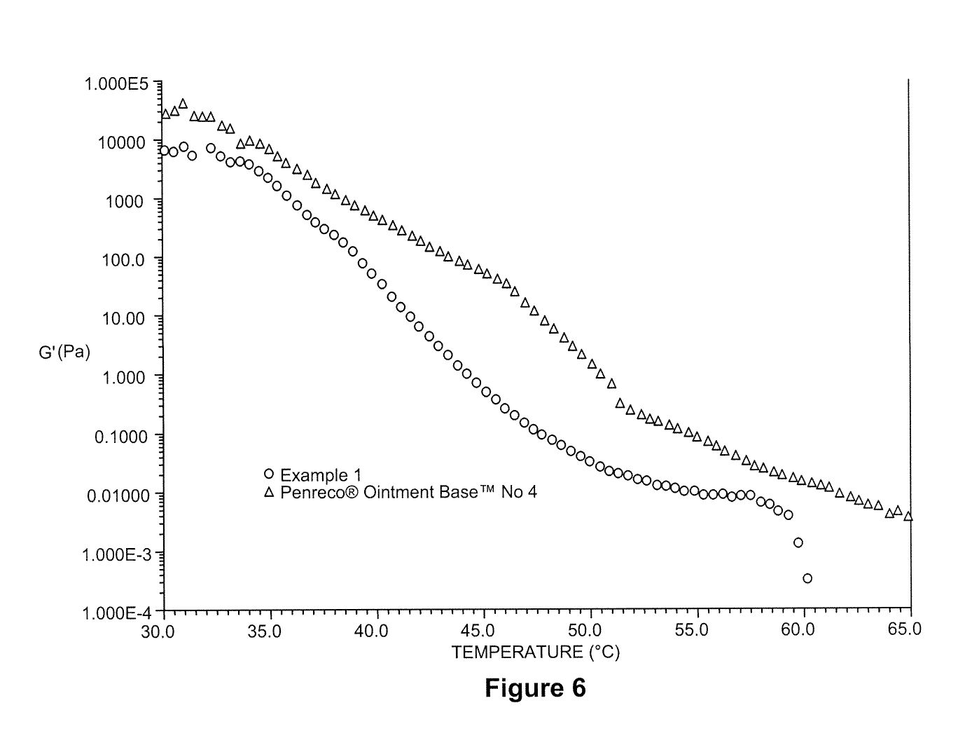

It is readily observed that the storage modulus for petrolatums of the present inventions at Pa=0.1 are substantially less than present day commercial petrolatums. FIG. 6 contains plots of G′(Pa) vs. temperature (° C.) showing the difference in storage modulus between the inventive petrolatum of Example 1 and a commercial petrolatum (Penreco® Ointment Base™ 4) having a similar melting point and consistency.

The complex viscosity analysis shows that the complex viscosity, i.e., |η*|(Pa·s=0.1), for present day commercial petrolatums is about 49.2° C. to greater than 65° C. In contrast, the inventive petrolatums have a |η*|(Pa·s=0.1) of about 45° C. or less Table VI summarizes the complex viscosities for petrolatums of the present invention and for present day commercial petrolatums. The data in Table VI show that an inventive petrolatum does not have a “draggy” feel, as compared to a present day commercial petrolatum.

TABLE VI

| | \|η*\| = 0.1 Pa · S | |
|---|---|---|
| Example 3 | 35.9° | C. |
| Example 1 | 36.3° | C. |
| Example 2 | 42.0° | C. |
| Ointment Base ™ No. 4 | 49.2° | C. |
| Snow White ™ Petrolatum USP | >65° | C. |
| Vaseline ® | >65° | C. |

Petrolatums of the present invention therefore have a complex viscosity |η*|(Pa·s=0.1) of about 45° C. or less, and typically about 35° C. to about 45° C. Preferred petrolatums have a |η*|(Pa·s=0.1) of about 35.5° C. to about 43° C.

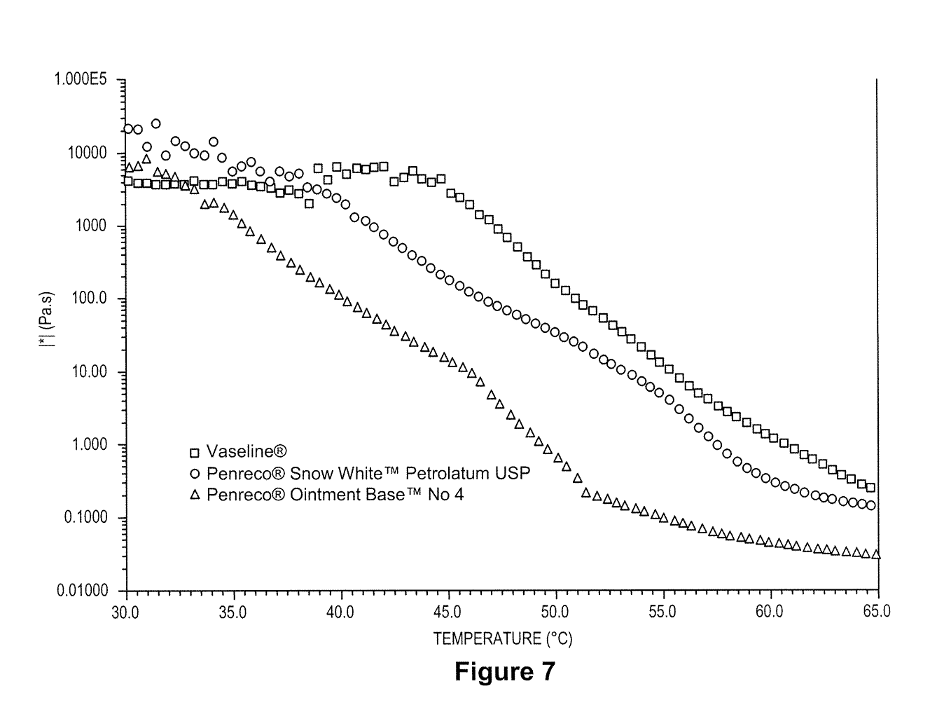

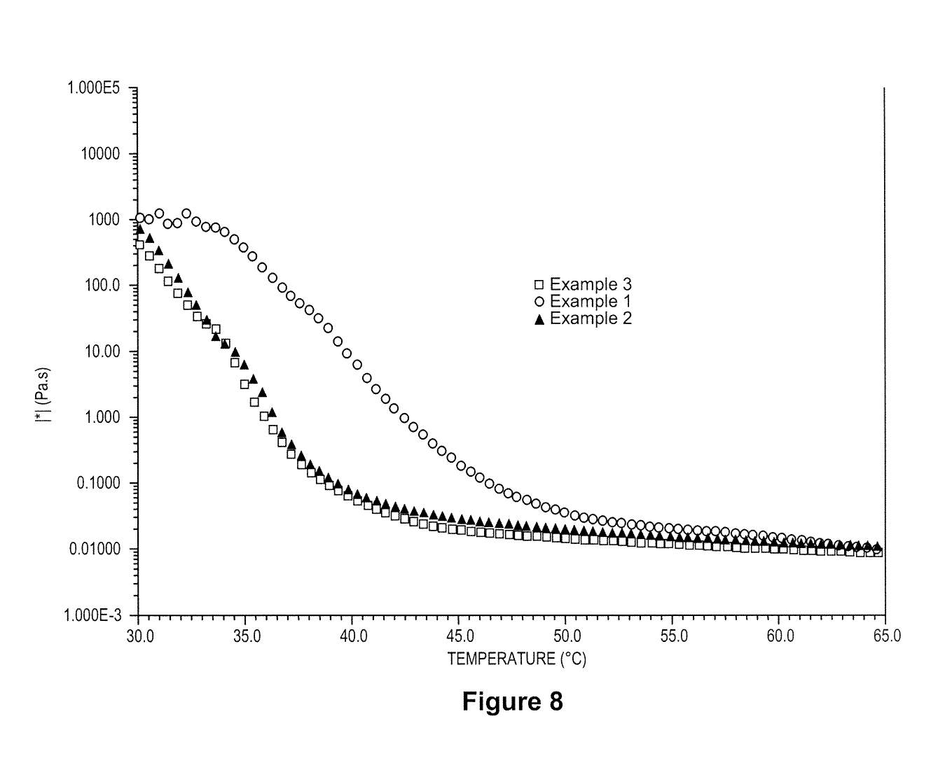

To further demonstrate the differences in complex viscosity between a present petrolatum and present day commercial petrolatums, FIGS. 7-9 contain plots of |η*|(Pa·s) vs. temperature (° C.). FIG. 7 contains plots for present day commercial petrolatums. FIG. 8 contains plots for petrolatums of the present invention.

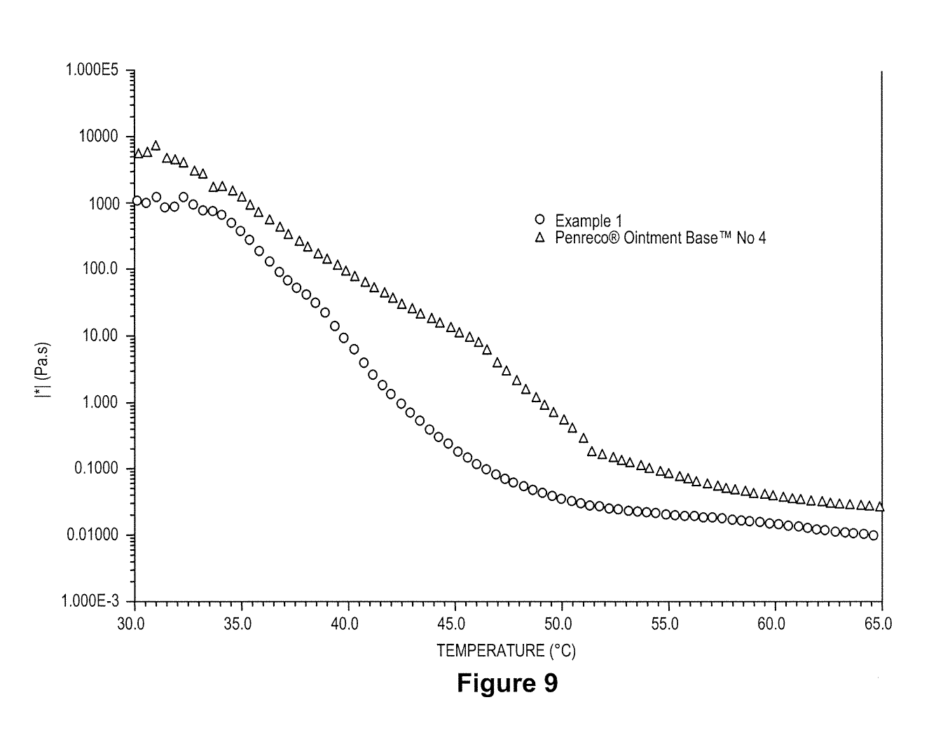

It is readily observed that the complex viscosity for petrolatums of the present invention at Pa·s=0.1 are substantially less than present day commercial petrolatums. FIG. 9 contains plots of |η*|(Pa·s) vs. temperature (° C.) showing the difference in complex viscosity between the petrolatum of Example 1 and a commercial petrolatum (Penreco® Ointment Base™ No. 4) having a similar melting point and consistency.

As stated above, it is theorized that the lower flow onset temperature and/or lower storage modulus and/or lower complex viscosity of the inventive petrolatums compared to similar commercial petrolatums at least partially contributes to the improved sensory properties exhibited by the present petrolatums. Unexpectedly, the sensory properties are improved to such an extent that the present petrolatums can be substituted for silicone fluids, in whole or in part, in cosmetic, personal care and pharmaceutical formulations. A present petrolatum also can be substituted for mineral oils often present in such formulations. No prior petrolatum could be substituted for a silicone fluid or a mineral oil, and exhibit the positive attributes of the silicone fluid or mineral oil.

Accordingly, a petrolatum of the present invention exhibits one or more of:

(a) a flow onset of about 45° C. or less;

(b) a storage modulus (G′=0.1 Pa) of about 50° C. or less; and

(c) a complex viscosity (|η*|(Pa·s=0.1) of about 45° C. or less.

Preferred petrolatums of the present invention exhibit at least two of (a), (b), and (c). More preferred inventive petrolatums exhibit each of (a), (b), and (c).

The above tests demonstrate that the present petrolatums exhibit the beneficial attributes of silicone fluids and differ from present day commercial petrolatums. However, it also is important for the present petrolatums to exhibit these beneficial attributes after incorporation into a personal care composition. It is well known that silicone fluids typically are not used in the “neat” form, but are introduced into formulations in order to obtain the beneficial attributes of the silicone fluid.

Accordingly, the following compositions A through L were prepared. The compositions are identical except for the presence of an inventive petrolatum of Examples 1-3 (Compositions A-C), a silicone fluid (Compositions D-I), or a present-day commercial petrolatum (Compositions J-L). Each composition was prepared identically by admixing the ingredients of Sequence 1, admixing the ingredients of Sequence 2, then adding the premixed ingredients of Sequence 2 to the premixed ingredients of Sequence 1 with stirring. The ingredient of Sequence 3 for each composition then was admixed with the admixture of Sequences 1 and 2, with stirring. Each composition contained 20.00% weight percent of a present petrolatum, a silicone fluid, or a present day commercial petrolatum, as noted in Table VII.

The following Table VII contains the weight percent of each ingredient and the specification for each composition.

TABLE VII

| | WEIGHT PERCENT OF INGREDIENT | | | | | | | | | | | | INGREDIENT | |
|---|---|---|---|---|---|---|---|---|---|---|---|---|---|---|
| SEQUENCE | A | B | C | D | E | F | G | H | I | J | K | L | (MANUFACTURER) | INCI NAME |
| 1 | 63.50 | 63.50 | 63.50 | 63.50 | 63.50 | 63.50 | 63.50 | 63.50 | 63.50 | 63.50 | 63.50 | 63.50 | Deionized Water | Water |
| 1 | 3.50 | 3.50 | 3.50 | 3.50 | 3.50 | 3.50 | 3.50 | 3.50 | 3.50 | 3.50 | 3.50 | 3.50 | Net LH | Hydrogenated Lecithin |
| | | | | | | | | | | | | | (Barnet Products Corp.) | (and) Sodium Methyl |
| | | | | | | | | | | | | | | Stearoyl Taurate (and) |
| | | | | | | | | | | | | | | Glycerin (and) |
| | | | | | | | | | | | | | | Squalane (and) |
| | | | | | | | | | | | | | | Hydroxypropyl- |
| | | | | | | | | | | | | | | methylcellulose |
| | | | | | | | | | | | | | | Stearoxy Ether |
| 2 | 1.00 | 1.00 | 1.00 | 1.00 | 1.00 | 1.00 | 1.00 | 1.00 | 1.00 | 1.00 | 1.00 | 1.00 | Botanistat PF-64 | Phenoxyethanol (and) |
| | | | | | | | | | | | | | (DD ChemCo) | Caprylyl Glycol (and) |
| | | | | | | | | | | | | | | Ethylhexylglycerin |
| | | | | | | | | | | | | | | (and) Hexylene Glycol |
| 2 | 6.00 | 6.00 | 6.00 | 6.00 | 6.00 | 6.00 | 6.00 | 6.00 | 6.00 | 6.00 | 6.00 | 6.00 | Keltrol (1%) (CP Kelco) | Water (and) Xanthan |
| | | | | | | | | | | | | | | Gum |
| 2 | 4.00 | 4.00 | 4.00 | 4.00 | 4.00 | 4.00 | 4.00 | 4.00 | 4.00 | 4.00 | 4.00 | 4.00 | Butylene Glycol | Butylene Glycol |
| | | | | | | | | | | | | | (Ruger Chemical) | |
| 3 | 1.00 | 1.00 | 1.00 | 1.00 | 1.00 | 1.00 | 1.00 | 1.00 | 1.00 | 1.00 | 1.00 | 1.00 | SS-10V | Sorbitan Stearate |
| | | | | | | | | | | | | | (Barnet Products Corp.) | |
| 3 | 1.00 | 1.00 | 1.00 | 1.00 | 1.00 | 1.00 | 1.00 | 1.00 | 1.00 | 1.00 | 1.00 | 1.00 | Behenyl Alcohol 80 | Behenyl Alcohol |
| | | | | | | | | | | | | | (Barnet Products Corp.) | |
| 3 | 20.00 | — | — | — | — | — | — | — | — | — | — | — | Example 3 | Petrolatum |
| 3 | — | 20.00 | — | — | — | — | — | — | — | — | — | — | Example 1 | Petrolatum |
| 3 | — | — | 20.00 | — | — | — | — | — | — | — | — | — | Example 2 | Petrolatum |
| 3 | — | — | — | 20.00 | — | — | — | — | — | — | — | — | Dow Corning ® | Cyclopentasiloxane |
| | | | | | | | | | | | | | 345 Fluid | and |
| | | | | | | | | | | | | | | Cyclohexasiloxane |
| 3 | — | — | — | — | 20.00 | — | — | — | — | — | — | — | Dow Corning ® | Dimethicone |
| | | | | | | | | | | | | | 200 Fluid, 50 cs | |
| 3 | — | — | — | — | — | 20.00 | — | — | — | — | — | — | Dow Corning ® | Dimethicone |
| | | | | | | | | | | | | | 200 Fluid, 100 cs | |
| 3 | — | — | — | — | — | — | 20.00 | — | — | — | — | — | Dow Corning ® | Dimethicone |
| | | | | | | | | | | | | | 200 Fluid, 200 cs | |
| 3 | — | — | — | — | — | — | — | 20.00 | — | — | — | — | Dow Corning ® | Dimethicone |
| | | | | | | | | | | | | | 200 Fluid, 350 cs | |
| 3 | — | — | — | — | — | — | — | — | 20.00 | — | — | — | Dow Corning ® | Dimethicone |
| | | | | | | | | | | | | | 200 Fluid, 500 cs | |
| 3 | — | — | — | — | — | — | — | — | — | 20.00 | — | — | Unilever Vaseline ® | Petrolatum |
| | | | | | | | | | | | | | Petroleum Jelly | |
| 3 | — | — | — | — | — | — | — | — | — | — | 20.00 | — | Penreco ® | Petrolatum |
| | | | | | | | | | | | | | Snow White ™ | |
| | | | | | | | | | | | | | Petrolatum, USP | |
| 3 | — | — | — | — | — | — | — | — | — | — | — | 20.00 | Penreco ® | Petrolatum |
| | | | | | | | | | | | | | Ointment Base ™ 4 | |

SPECIFICATIONS:
LVT-E @ 1.5 rpm (1 minute) cps

| | 118,000 | 118,700 | 131,500 | 265,000 | 103,700 | 75,620 | 76,250 | 76,870 | 136,200 | 72,800 | 76,560 | 100,900 |
|---|---|---|---|---|---|---|---|---|---|---|---|---|
| pH | 6.83 | 6.99 | 6.92 | 6.95 | 6.87 | 7.1 | 6.9 | 6.93 | 6.87 | 6.78 | 6.91 | 6.87 |

Table VIII provides a comparison of Compositions A-L for the various properties that contribute to “afterfeel”. In this test, Compositions A-L were tested by a panel of trained judges using the protocol of ASTM E1490. Table VIII shows that commercial present day petrolatums (Compositions J-L) provided compositions having a draggy and tacky feel, and failed to exhibit any of the positive afterfeel attributes typically provided by a silicone fluid (Compositions D-I). In contrast, the inventive petrolatums (Compositions A-C) provided compositions that exhibit the positive afterfeel attributes of a silicone fluid.

TABLE VIII
COMPARISON OF SENSORY ATTRIBUTES IN A FORMULATION

| | | | | Silicones (Dow Corning ®) | | |
|---|---|---|---|---|---|---|
| | | | | Dow Corning ® 345 | Dow Corning ® | Dow Corning ® |
| | | | | Fluid, | 200 Fluid, | 200 Fluid, |
| | Inventive Petrolatum | | | Cyclopentasiloxane | 50 cs | 100 cs |
| | Example 3 | Example 1 | Example 2 | (and) | Dimethicone | Dimethicone |
| | (Composition | (Composition | (Composition | Cyclohexasiloxane | (Composition | (Compositon |
| | A) | B) | C) | (Composition D) | E) | F) |
| Light | ✓ | | | ✓ | ✓ | ✓ |
| Fast Absorption | ✓ | | | ✓ | ✓ | |
| Smooth | | ✓ | ✓ | | | |
| Soft | | ✓ | | | | ✓ |
| Cushiony | | | ✓ | | | |
| Draggy | | | | | | |
| Tacky | | | | | | |

| | Silicones (Dow Corning ®) | | | Commercial USP Petrolatums | | |
|---|---|---|---|---|---|---|
| | Dow Corning ® | Dow Corning ® | Dow Corning ® | Unilever | Penreco ® | |
| | 200 Fluid, | 200 Fluid, | 200 Fluid, | Vaseline ® | Snow White ™ | Penreco ® |
| | 200 cs | 350 cs | 500 cs | Petroleum | Petrolatum, | Ointment |
| | Dimethicone | Dimethicone | Dimethicone | Jelly | USP | Base ™ 4 |
| | (Composition | (Composition | (Composition | (Composition | (Composition | (Composition |
| | G) | H) | I) | J) | K) | L) |
| Light | | | | | | |
| Fast Absorption | | | | | | |
| Smooth | ✓ | ✓ | ✓ | | | |
| Soft | ✓ | | | | | |
| Cushiony | | ✓ | ✓ | | | |
| Draggy | | | | ✓ | ✓ | ✓ |
| Tacky | | | | ✓ | ✓ | ✓ |

Table VII confirms the findings summarized in Table III. In particular, that the petrolatum of Example 3 is an excellent substitute for cyclopentasiloxane, cyclohexasiloxane, and 50 cs and 100 cs dimethicones; Example 1 is an excellent substitute for 200 cs dimethicone; and Example 2 is an excellent substitute for 350 cs and 500 cs dimethicones.

Table IX contains additional comparisons between Compositions A-L, and an additional Composition M, which shows that a blend of inventive petrolatums can provide afterfeel attributes of silicone fluids that are not available commercially.

TABLE IX

| Composition | Evaluation |
|---|---|
| Composition A | close in feel to Composition E (50 cs dimethicone), |
| | dry, light |
| Composition D | very light, dry, fast absorbing |
| Composition E | not as light, absorbs fast, but has more play time than |
| | Composition D |
| Composition B | close in feel to Composition G (200 cs dimethicone) |
| Composition F | lighter, powdery feel |
| Composition G | soft afterfeel |
| Composition C | cushiony feel, falls between Composition H (350cs) |
| | and Composition I (500cs), dimethicone fluids between |
| | 350cs and 500cs are not available commercially |
| Composition H | lighter cushion |
| Composition I | heavier cushion |
| Composition J | same application as Composition K, very tacky |
| | and draggy afterfeel |
| Composition K | better slip than Composition J, very tacky and |
| | draggy afterfeel |
| Composition L | heavy, draggy application, more residue, greasy |
| | and shiny |

The present petrolatums therefore allow the preparation of silicone-free cosmetic, personal care, and pharmaceutical compositions that exhibit the identical, or essentially identical, sensory properties as an identical composition containing a silicone fluid as a full and direct substitute for the petrolatum. For example, a composition containing a petrolatum of Example 3 exhibits the same sensory properties as a composition containing an identical amount of corresponding Dow Corning® 345 Fluid or a corresponding dimethicone (50 cs) and essentially the same sensory properties as a composition containing an identical amount of corresponding dimethicone (100 cs), as a substitute for the petrolatum.

A composition containing the petrolatum of Example 1 exhibits the same sensory properties as a composition containing an identical amount of a corresponding dimethicone (200 cs) and essentially the same sensory properties as a composition containing an identical amount of a corresponding dimethicone (350 cs) or a corresponding dimethicone (500 cs), as a substitute for the petrolatum.

A composition containing the petrolatum of Example 2 exhibits the same sensory properties as a composition containing an identical amount of a corresponding dimethicone (350 cs) or a corresponding dimethicone (500 cs) and essentially the same sensory properties as a composition containing an identical amount of a corresponding dimethicone (200 cs).

The term “same sensory properties” therefore means that a composition containing a present petrolatum exhibits the same sensory properties listed in Table VIII, and determined by a panel of trained judges using the protocols of ASTM Method E1490, as a composition containing a silicone fluid that corresponds to the petrolatum, as set forth in Table VIII and above. The term “essentially the same sensory properties” means that a composition containing a present petrolatum exhibits at least one of the same sensory properties listed in Table VIII, and determined by a panel of trained judges using the protocols of ASTM Method E1490, as a composition containing a silicone fluid that corresponds to the petrolatum, as set forth in Table VIII and above.

The present petrolatums provide superior skin protection and provide the feel of a silicone. The present petrolatums therefore can be formulated into a variety of personal care, cosmetic, and pharmaceutical compositions as substitutes for silicone fluids. The present petrolatums also can be substituted, wholly or in part, for mineral oil. The present petrolatums meet USP and FDA requirements and can be used, for example, as an emollient, conditioner, moisturizer, and ointment base. The present petrolatums can be formulated into hand and body creams and lotions, liquid soaps, lip balm, hair conditioners, scalp protectors, body washes, massage creams, sunscreens, tanning products, shaving products, cuticle moisturizers, and pomades, for example.

The present petrolatums have an excellent solubility in a variety of organic compounds and silicone fluids that typically are found in cosmetic, personal care, and pharmaceutical compositions. For example, the petrolatums of Examples 1, 2, and 3 are soluble (i.e., a clear solution at 55° C.) at 30% by weight in caprylic/capric triglycerides, isopropyl palmitate, sunflower seed oil, soybean oil, dimethicone (20 cs), and cyclopentasiloxane; and are miscible at 30% by weight in dimethicone (350 cs).

The present petrolatums liquefy on skin contact, and compared to present day commercial petrolatums have a silkier, smoother afterfeel, less drag, low tack, excellent spreadability, easier handling, and are faster absorbing.

The following are additional examples of compositions containing a petrolatum of the present invention.

### Example 1

Moisturizing Hair Conditioner

| | Percent | | |
|---|---|---|---|
| Sequence | (wt %) | Ingredient | INCI Name |
| 1 | 79.50 | Deionized Water | Water |
| 1 | 0.10 | DISSOLVENE Na2 | Disodium EDTA |
| | | (Akzo Nobel) | |
| 1 | 0.25 | BOTANISTAT PF-64 | Phenoxyethanol (and) |
| | | (DD ChemCo) | Caprylyl |
| | | | Glycol (and) |
| | | | Ethylhexylglycerin (and) |
| | | | Hexylene Glycol |
| 1 | 3.50 | Glycerin | Glycerin |
| | | (Ruger Chemical Co.) | |
| 1 | 5.00 | INCROQUAT Behenyl | Behentrimonium |
| | | TMC-85 | Chloride |
| | | (Croda, Inc.) | |
| 2 | 5.50 | Example 2 | Petrolatum |
| | | | (inventive petrolatum) |
| 2 | 0.25 | LIPOVOL WGO | Triticum Vulgare |
| | | (Lipo Chemicals, Inc.) | (Wheat) |
| | | | Germ Oil |
| 2 | 0.10 | Vitamin E Acetate | Tocopheryl Acetate |
| | | (BASF) | |
| 2 | 2.00 | LIPOCOL C | Cetyl Alcohol |
| | | (Lipo Chemicals, Inc.) | |
| 2 | 3.00 | LIPOWAX P | Cetearyl Alcohol (and) |
| | | (Lipo Chemicals, Inc.) | Polysorbate 60 |
| 3 | 0.30 | JEEN DMDM Hydantoin | DMDM Hydantoin |
| | | (Jeen International) | |
| 4 | 0.50 | HYDROTRITICUM | Hydrolyzed Wheat |
| | | 2000 (Croda, Inc.) | Protein |
| 5 | qs | Citric Acid (50%) | Citric Acid |
| | | (Ruger Chemical Co.) | |

Manufacturing Procedure:
1. Mix and heat Sequence 1 components to 80° C. with medium speed propeller mixing.
2. Combine Sequence 2 components and heat to 80° C. Mix until all waxes are melted.
3. Slowly add Sequence 2 to Sequence 1 and mix for 15 minutes. Begin cooling.
4. At 30° C., add Sequence 3 component and Sequence 4 component and mix well.
5. Adjust pH to 3.5-3.7 with Sequence 5 component.
Specifications:
pH: 3.56
Viscosity: LV T-D @ 0.3 rpm 371,200 cps

### Example 2

Skincare Emulsion

| | Percent | | |
|---|---|---|---|
| Sequence | wt % | Ingredient | INCI Name |
| 1 | 5.00 | Example 3 | Petrolatum |
| | | | (inventive petrolatum) |
| 1 | 10.00 | DC 5225C | Cyclopentasiloxane (and) |
| | | (Dow Corning) | PEG/PPG-18/18 |
| | | | Dimethicone |
| 1 | 1.00 | Example 1 | Petrolatum |
| 2 | 62.15 | Deionized Water | Water |
| 2 | 0.10 | DISSOLVENE Na2 | Disodium EDTA |
| | | (Akzo Nobel) | |
| 2 | 0.75 | LIPOSERV PP | Phenoxyethanol (and) |
| | | (Lipo Chemicals, Inc.) | Methylparaben (and) |
| | | | Ethylparaben |
| | | | (and) Butylparaben (and) |
| | | | Propylparaben (and) |
| | | | Isobutylparaben |
| 2 | 18.00 | Butylene Glycol | Butylene Glycol |
| | | (Ruger Chemical Co.) | |
| 2 | 3.00 | NaCl | Sodium Chloride |

Procedure:
1. Combine Sequence 1 components and heat to 40° C. with high speed propeller mixing. Mix until homogenous.
2. Combine Sequence 2 components and heat to 80° C. Mix until free of solid particles and cool to 40° C.
3. Very slowly add Sequence 2 components to Sequence 1 and mix with high speed propeller mixing.
4. Homogenize for 1 minute.
Specifications:
Viscosity: LV T-E @ 0.6 rpm 581,000 cps-703,000 cps

### Example 3

Oil Free SPF Lotion

| | Percent | | |
|---|---|---|---|
| Sequence | wt % | Ingredient | INCI Name |
| 1 | 2.10 | LIPOMULSE 165 | Glyceryl Stearate (and) PEG-100 Stearate |
| | | (Lipo Chemicals) | |
| 1 | 0.90 | MONTANOV S | Coco-Glucoside (and) Coconut Alcohol |
| | | (Seppic) | |
| 1 | 15.00 | Example 3 | Petrolatum (inventive) |
| 1 | 3.00 | NEO HELIOPAN 357 | Butyl Methoxydibenzoylmethane |
| | | (Symrise) | |
| 1 | 5.00 | NEO HELIOPAN OS | Octyl Salicylate |
| | | (Symrise) | |
| 1 | 4.00 | CORAPAN TQ | Diethylhexyl 2,6-Naphthalate |
| | | (Symrise) | |
| 1 | 1.50 | SOFTISAN 100 | Hydrogenated Coco Glycerides |
| | | (Sasol) | |
| 1 | 0.10 | BISABOLOL | Bisabolol |
| | | (Lipo Chemicals) | |
| 2 | 1.50 | SEPIGEL 305 | Polyacrylamide (and) C13-14 Isoparaffin |
| | | (Seppic) | (and) Laureth-7 |
| 3 | 60.55 | Deionized Water | Water |
| 3 | 1.00 | BOTANISTAT PF-64 | Phenoxyethanol (and) Caprylyl Glycol |
| | | (D-D Chemco) | (and) Ethylhexylglycerin (and) Hexylene |
| | | | Glycol |
| 3 | 5.00 | Glycerin | Glycerin |
| 3 | 0.10 | DISSOLVINE Na2 | Disodium EDTA |
| | | (Akzo Nobel) | |
| 3 | 0.25 | Actiphyte of Aloe | Butylene Glycol (and) Water (and) Aloe |
| | | BG50 | Barbadensis Leaf Extract |
| | | (Active Organics) | |

Procedure:
1. Combine Sequence 1 components with moderate speed propeller mixing. Heat to 78-80° C.
2. Add Sequence 2 component and mix until uniform.
3. Combine Sequence 3 components, heat to 80° C. with propeller mixing. Mix until clear.
4. Slowly add Sequence 3 to combined Sequence 1 and Sequence 2, and mix for 15 minutes.
5. Cool to 25° C.
Specifications:
pH: 4.98-5.02
Viscosity: LVT #4 @ 12 rpm 30,200 cps

### Example 4

Clear Ringing Gel

| | Percent | | |
|---|---|---|---|
| Sequence | wt % | Ingredient | INCI Name |
| 1 | 56.99 | Deionized Water | Water |
| 1 | 1.00 | LIPOSERV PP | Phenoxyethanol (and) |
| | | (Lipo Chemicals, Inc.) | Methylparaben (and)Ethylparaben |
| | | | (and) Butylparaben (and) |
| | | | Propylparaben (and) |
| | | | Isobutylparaben |
| 1 | 12.00 | Butylene Glycol | Butylene Glycol |
| | | (Ruger Chemical Co.) | |
| 1 | 0.01 | FD&C Red #33 | FD&C Red #33 |
| | | (1% aq. Solution) | |
| | | (Warner Jenkinson Co., Inc.) | |
| 2 | 6.00 | CRODAFOS N-10 Neutral | DEA-Oleth-10 Phosphate |
| | | (Croda, Inc.) | |
| 2 | 5.00 | LIPOCOL O-5 | Oleth-5 |
| | | (Lipo Chemicals, Inc.) | |
| 2 | 4.00 | LIPOCOL O-3 | Oleth-3 |
| | | (Lipo Chemicals, Inc.) | |
| 2 | 15.00 | Example 3 | Petrolatum (inventive petrolatum) |

Procedure:
1. Combine Sequence 1 components and heat to 85° C.-87° C. with medium speed propeller mixing.
2. Combine Sequence 2 components and heat to 85° C.-87° C. Mix until clear with medium/low speed propeller mixing.
3. Slowly add Sequence 2 to Sequence 1 and mix until smooth.
Specifications:
pH: 6.60
Viscosity: LV T-E @ 0.3 rpm 1,357,000 cps

What is claimed is:

1. A petrolatum having properties of:
(a) a drop melting point (ASTM D127) of about 100° F. to about 125° F.;
(b) a cone penetration (ASTM D937) of about 250 to about 300 dmm;
(c) a dynamic viscosity at 25° C. of about 100,000 to about 200,000 centipoise, and
(d) at least one of
(i) a flow onset of about 45° C. or less;
(ii) a storage modulus at 0.1 Pa of about 50° C. or less, and
(iii) a complex viscosity at 0.1 Pa·S of about 45° C. or less.

2. The petrolatum of claim 1 comprising
about 5% to about 20% saturated straight chain hydrocarbons with a carbon number distribution of about C₂₀-C₄₅;
0% to about 20% saturated microcrystalline hydrocarbons with a carbon number distribution of about C₃₀-C₉₀; and
about 60% to about 85% saturated branched and cycloparaffinic hydrocarbons within a carbon number distribution of about C₁₀-C₅₀.

3. The petrolatum of claim 1 having at least two of (i), (ii), and (iii).

4. The petrolatum of claim 1 having each of (i), (ii), and (iii).

5. The petrolatum of claim 1 further having the properties of:
congealing point (ASTM D938) about 90° F. to about 98° F.; and
average molecular weight (ASTM D2502) about 300 to about 450.

6. The petrolatum of claim 1 having a drop melting point of about 105° F. to about 120° F.

7. The petrolatum of claim 1 having a cone penetration of about 270 to about 300 dmm.

8. The petrolatum of claim 1 having a dynamic viscosity of about 125,000 to about 175,000 centipoise.

9. The petrolatum of claim 1 having a flow onset of about 30° C. to about 45° C.

10. The petrolatum of claim 1 having a storage modulus of about 35° C. to about 50° C.

11. The petrolatum of claim 1 having a complex viscosity of about 35° C. to about 45° C.

12. The petrolatum of claim 1 having the properties of

| | | |
|---|---|---|
| Congealing Point | (ASTM D938) | about 95 |
| Drop Melting Point | (ASTM D127) | about 108 |
| Cone Penetration | (ASTM D937) | about 298 |
| Viscosity @ 25° C. | (Dynamic Viscometer) | about 150,000 cps |

13. The petrolatum of claim 12 further having one or more of a flow onset of about 40° C., a storage module of about 48° C., and a complex viscosity of about 36.5° C.

14. The petrolatum of claim 12 further having the properties of

| | | |
|---|---|---|
| Lovibond Color, 3″ cell | (IP 17) | about 0.6 |
| Viscosity @ 130° F., cSt | (ASTM D445) | about 11.3 |
| Viscosity @ 210° F., cSt | (ASTM D445) | about 4 |
| Specific Gravity @ 60° C./25° C. | (ASTM D1298) | about 0.82 |
| Saybolt Color | (ASTM D156) | about 24 |
| Carbon Number @ 5% Point | (IP 507-4) | about 19 |
| Average Molecular Weight | (ASTM D2502) | about 365 |
| Refractive Index @ 130° F. | (ASTM D1218) | about 1.454 |

15. The petrolatum of claim 1 having the properties of

| | | |
|---|---|---|
| Congealing Point | (ASTM D938) | about 93 |
| Drop Melting Point | (ASTM D127) | about 105 |
| Cone Penetration | (ASTM D937) | about 273 |
| Viscosity @ 25° C. | (Dynamic Viscometer) | about 150,000 cps |

16. The petrolatum of claim 15 further having one or more of a flow onset of about 37° C., a storage module of about 39.5° C., and a complex viscosity of about 42° C.

17. The petrolatum of claim 15 further having the properties of

| | | |
|---|---|---|
| Lovibond Color, 3″ cell | (IP 17) | about 0.3 |
| Viscosity @ 130° F., cSt | (ASTM D445) | about 11.9 |
| Viscosity @ 210° F., cSt | (ASTM D445) | about 4.4 |
| Specific Gravity @ 60° C./25° C. | (ASTM D1298) | about 0.82 |
| Saybolt Color | (ASTM D156) | about 26 |
| Carbon Number @ 5% Point | (IP 507-4) | about 19 |
| Average Molecular Weight | (ASTM D2502) | about 410 |
| Refractive Index @ 130° F. | (ASTM D1218) | about 1.456 |

18. The petrolatum of claim 1 having the properties of:

| | | |
|---|---|---|
| Congealing Point | (ASTM D938) | about 92 |
| Drop Melting Point | (ASTM D127) | about 115 |
| Cone Penetration | (ASTM D937) | about 278 |
| Viscosity @ 25° C. | (Dynamic Viscometer) | about 150,000 cps |

19. The petrolatum of claim 18 having one or more of a flow onset of about 32° C., a storage modulus of about 39° C., and a complex viscosity of about 36° C.

20. The petrolatum of claim 18 further having the properties of

| | | |
|---|---|---|
| Lovibond Color, 3″ cell | (IP 17) | about 0.2 |
| Viscosity @ 130° F., cSt | (ASTM D445) | about 11 |
| Viscosity @ 210° F., cSt | (ASTM D445) | about 4 |
| Specific Gravity @ 60° C./25° C. | (ASTM D1298) | about 0.82 |
| Saybolt Color | (ASTM D156) | about 30 |
| Carbon Number @ 5% Point | (IP 507-4) | about 15 |
| Average Molecular Weight | (ASTM D2502) | about 380 |
| Refractive Index @ 130° F. | (ASTM D1218) | about 1.452 |

21. A composition comprising a petrolatum of claim 1 and a carrier, wherein the composition is free of a silicone.

22. The composition of claim 21 wherein the carrier comprises water.

23. The composition of claim 21 wherein after an application of the composition to skin or hair, the skin or hair exhibits essentially identical sensory properties as skin or hair treated with an identical composition containing a corresponding silicone fluid as a substitute for the petrolatum of claim 1, and in the same weight amount as the petrolatum of claim 1.

24. The composition of claim 23 wherein the petrolatum has properties of

| | | |
|---|---|---|
| Congealing Point | (ASTM D938) | about 95 |
| Drop Melting Point | (ASTM D127) | about 108 |
| Cone Penetration | (ASTM D937) | about 298 |
| Lovibond Color, 3″ cell | (IP 17) | about 0.6 |
| Viscosity @ 130° F., cSt | (ASTM D445) | about 11.3 |
| Viscosity @ 210° F., cSt | (ASTM D445) | about 4 |
| Specific Gravity @ 60° C./25° C. | (ASTM D1298) | about 0.82 |
| Saybolt Color | (ASTM D156) | about 24 |
| Carbon Number @ 5% Point | (IP 507-4) | about 19 |
| Average Molecular Weight | (ASTM D2502) | about 365 |
| Refractive Index @ 130° F. | (ASTM D1218) | about 1.454 |
| Viscosity @ 25° C. | (Dynamic | about 150,000 cps |
| | Viscometer) | |
| Flow Onset | | about 31.7° C. |
| Storage Modulus at 0.1 Pa | | about 47.4° C. |
| Complex Viscosity at 0.1 Pa · s | | about 36.3° C. |

and the corresponding silicone fluid is a dimethicone (200 centistokes).

25. The composition of claim 23 wherein the petrolatum has properties of

| | | |
|---|---|---|
| Congealing Point | (ASTM D938) | about 93 |
| Drop Melting Point | (ASTM D127) | about 105 |
| Cone Penetration | (ASTM D937) | about 273 |
| Lovibond Color, 3″ cell | (IP 17) | about 0.3 |
| Viscosity @ 130° F., cSt | (ASTM D445) | about 11.9 |
| Viscosity @ 210° F., cSt | (ASTM D445) | about 4.4 |
| Specific Gravity @ 60° C./25° C. | (ASTM D1298) | about 0.82 |
| Saybolt Color | (ASTM D156) | about 26 |
| Carbon Number @ 5% Point | (IP 507-4) | about 19 |
| Average Molecular Weight | (ASTM D2502) | about 410 |
| Refractive Index @ 130° F. | (ASTM D1218) | about 1.456 |
| Viscosity @ 25° C. | (Dynamic | about 150,000 cps |
| | Viscometer) | |
| Flow Onset | | about 36.7° C. |
| Storage Modulus at 0.1 Pa | | about 39.4° C. |
| Complex Viscosity at 0.1 Pa · s | | about 42.0° C. |

and the corresponding silicone fluid is a dimethicone (350 centistoke), a dimethicone (500 centistoke), or a mixture thereof.

26. The composition of claim 23 wherein the petrolatum has properties of

| | | |
|---|---|---|
| Congealing Point | (ASTM D938) | about 92 |
| Drop Melting Point | (ASTM D127) | about 115 |
| Cone Penetration | (ASTM D937) | about 278 |
| Lovibond Color, 3″ cell | (IP 17) | about 0.2 |
| Viscosity @ 130° F., cSt | (ASTM D445) | about 11 |
| Viscosity @ 210° F., cSt | (ASTM D445) | about 4 |
| Specific Gravity @ 60° C./25° C. | (ASTM D1298) | about 0.82 |
| Saybolt Color | (ASTM D156) | about 30 |
| Carbon Number @ 5% Point | (IP 507-4) | about 15 |
| Average Molecular Weight | (ASTM D2502) | about 380 |
| Refractive Index @ 130° F. | (ASTM D1218) | about 1.452 |
| Viscosity @ 25° C. | (Dynamic | about 150,000 cps |
| | Viscometer) | |
| Flow Onset | | about 31.7° C. |
| Storage Modulus at 0.1 Pa | | about 39.0° C. |
| Complex Viscosity at 0.1 Pa · s | | about 35.9° C. |

and the corresponding silicone fluid is a cyclopentasiloxane, a cyclohexasiloxane, a dimethicone (50 centistokes), a dimethicone (100 centistokes), or a mixture thereof.
